(19) United States
(12) Patent Application Publication
(10) Pub. No.: US 2026/0176523 A1
(43) Pub. Date: Jun. 25, 2026

(54) METHOD FOR IN-SITU REMOVAL OF RESERVOIR PLUGGING USING MICROORGANISMS AND IN-SITU RESERVOIR OIL DISPLACEMENT, AND IRON-REDUCING BACTERIUM ACTIVATOR

(71) Applicants: CHINA UNIVERSITY OF GEOSCIENCES BEIJING, Beijing (CN); YANGTZE UNIVERSITY, Wuhan, Hubei (CN)

(72) Inventors: Fan ZHANG, Beijing (CN); Yuehui SHE, Beijing (CN); Qing FENG, Beijing (CN); Shanshan Sun, Beijing (CN); Xiaonan LI, Beijing (CN); Hao DONH, Beijing (CN); Gaoming YU, Beijing (CN)

(21) Appl. No.: 18/832,163

(22) PCT Filed: May 26, 2023
(86) PCT No.: PCT/CN2023/096442

§ 371 Date: Jul. 23, 2024

(30) Foreign Application Priority Data
Apr. 24, 2023 (CN) 202310445534.4

(51) Int. Cl. C09K 8/582

(52) U.S. Cl. CPC C09K 8/582; C09K 2208/10; C09K 2208/24

(57) ABSTRACT

The present application belongs to the technical field of petroleum extraction, and specifically relates to a method for in-situ removal of reservoir plugging using microorganisms and in-situ reservoir oil displacement, and an iron-reducing bacterium activator. The method includes the following steps: injecting a microbial mixture into a target reservoir to allow in-situ activation of microorganisms, such that magnetic iron nanoparticles are generated in-situ in the target reservoir to obtain a plugging-removed target reservoir; wherein the microbial mixture includes an iron-reducing bacterium fermentation broth and an iron-reducing bacterium activator; and injecting water into the plugging-removed target reservoir to allow the reservoir oil displacement. In summary, the method removes reservoir plugging caused by secondary precipitation of Fe(OH)₃ through an environmental-friendly biological method. Meanwhile, the microorganisms with an iron-reducing activity enable a green and low-cost biological method to produce highly-active magnetic nanoparticles in situ to allow the reservoir oil displacement.

The present application claims priority to the Chinese Patent Application No. CN202310445534.4, filed with the China National Intellectual Property Administration (CNIPA) on Apr. 24, 2023, and entitled “METHOD FOR IN-SITU REMOVAL OF RESERVOIR PLUGGING USING MICROORGANISMS AND IN-SITU RESERVOIR OIL DISPLACEMENT, AND IRON-REDUCING BACTERIUM ACTIVATOR”, which is incorporated herein by reference in its entirety.

### TECHNICAL FIELD

The present disclosure belongs to the technical field of petroleum extraction, and specifically relates to a method for in-situ removal of reservoir plugging using microorganisms and in-situ reservoir oil displacement, and an iron-reducing bacterium activator.

### BACKGROUND

There are large amounts of iron-containing minerals in crude oil reservoirs. Sedimentary stratigraphic studies show that iron-containing carbonates and associated minerals are mainly pore-filling materials, forming dense cement between pores during the formation compaction evolution. These substances are a main cause of low permeability in reservoirs. Since these iron-containing substances are sensitive to acid, acidification plugging removal has become a conventional method to improve the pore structure of reservoirs and is widely used in many oil fields. However, these iron-containing substances inevitably produce amorphous Fe(OH)₃ precipitation during the acidification, causing a secondary precipitation damage to the reservoirs. Moreover, with the progress of oil field development, especially water injection development, surface pipelines and oil reservoirs form an injection-production cycle system, causing surface metal pipelines to suffer varying degrees of corrosion. The amorphous Fe(OH)₃ precipitation formed by free Fe³⁺ ions during the corrosion becomes another main component of inorganic scale plugging in the reservoir. Therefore, the presence of a large amount of amorphous Fe(OH)₃ in the reservoir is one of the main reasons for reservoir plugging. People try to remove the plugging through various approaches in order to establish smooth oil, gas, and water channels in the reservoir.

Furthermore, nanofluid as an efficient oil displacement agent has been used in oil fields to enhance oil recovery factor. Compared with other nanomaterials, iron nanofluid is considered to be the most intelligent nanofluid due to unique magnetic characteristics. Although showing certain advantages in improving the oil recovery factor, the iron nanofluid also has certain limitations during the application in actual mines. On one hand, the synthesis or modification of industrial metal nanomaterials by physical and chemical methods has complex processes and high costs, which are easy to produce by-products that affect the environment and are harmful to human health. On the other hand, traditional nano-oil displacement mines adopt industrially-synthesized nanomaterials on the ground to form a nano-dispersion system by adding chemical reagents such as dispersants, and then inject the nano-dispersion system into a reservoir through the injection system. Accordingly, dispersion stability, formation adsorption loss, and formation compatibility of the nanomaterials can greatly affect a formation sweep coefficient and an oil displacement effect of the nano-oil displacement agents.

In summary, current oil production suffers from serious reservoir plugging, poor oil displacement effect, and high cost.

### SUMMARY

An objective of the present disclosure is to provide a method for in-situ removal of reservoir plugging using microorganisms and in-situ reservoir oil displacement, and an iron-reducing bacterium activator. In the present disclosure, the method can relieve reservoir plugging damages caused by Fe(OH)₃, and can prepare magnetic iron nanoparticles in a green and low-cost manner to achieve efficient oil displacement.

To achieve the above objective, the present disclosure provides the following technical solutions:

The present disclosure provides a method for in-situ removal of reservoir plugging using microorganisms and in-situ reservoir oil displacement, including the following steps:
injecting a microbial mixture into a target reservoir to allow in-situ activation of microorganisms, such that magnetic iron nanoparticles are generated in-situ in the target reservoir to obtain a plugging-removed target reservoir; where the microbial mixture includes an iron-reducing bacterium fermentation broth and an iron-reducing bacterium activator; and the iron-reducing bacterium activator includes NH₄Cl, NaCl, KCl, molasses, HCOONa, CoSO₄, Na₂MoO₄, NiCl₂, Na₂WO₄, and H₃BO₃; and
injecting water into the plugging-removed target reservoir to allow the reservoir oil displacement.

Preferably, the iron-reducing bacterium fermentation broth includes an iron-reducing bacterium; and
the iron-reducing bacterium is one or more selected from the group consisting of Shewanella chilikensis, Bacillus alkalitelluris, Tessaracoccus profundi, Tessaracoccus oleiagri, Clostridium swellfunianum, and Deferribacter thermophilus.

Preferably, the iron-reducing bacterium activator includes the following components in parts by mass:
10 parts to 15 parts of the NH₄Cl, 5 parts to 10 parts of the NaCl, 5 parts to 10 parts of the KCl, 10 parts to 20 parts of the molasses, 40 parts to 60 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃.

Preferably, a preparation process of the iron-reducing bacterium fermentation broth includes the following steps:
inoculating the iron-reducing bacterium into a medium with an inoculum size of 5% to allow anaerobic fermentation at 37° C. for 5 d to obtain the iron-reducing bacterium fermentation broth; where the medium includes corn steep liquor, molasses, HCOONa, (NH₄)₂HPO₄, and NaCl.

Preferably, the iron-reducing bacterium in the iron-reducing bacterium fermentation broth has a bacterial count of 5×10⁷ cells/mL to 8×10⁷ cells/mL; and
the microbial mixture includes 5% to 10% of the iron-reducing bacterium fermentation broth and 5% to 10% of the iron-reducing bacterium activator by mass percent.

Preferably, in the medium, a dry weight of the corn steep liquor has a mass percent of 3%; the molasses has a mass percent of 3%; the HCOONa has a mass percent of 5%; the (NH₄)₂HPO₄ has a mass percent of 0.1%; the NaCl has a mass percent of 1%; and the medium has a pH value of 7.2.

Preferably, the microbial mixture is injected at an injection volume of 15 PV to 20 PV; the PV represents a pore volume, and a calculation formula of the PV is shown in Formula 1:
1PV=π×R²×H×Ø Formula 1; in Formula 1, R represents a treatment radius of the microbial mixture, in m; H represents a thickness of a water-absorbing layer, in m; and Ø represents an average porosity of a formation.

Preferably, the microbial mixture is injected at an injection rate of (0.1-0.5) m³/min; and
the in-situ activation of the microorganism is conducted for 7 d to 10 d.

Preferably, the water is injected at a flow rate of (0.1-0.5) m³/min.

The present disclosure further provides an iron-reducing bacterium activator, including the following components in parts by mass:
10 parts to 15 parts of the NH₄Cl, 5 parts to 10 parts of the NaCl, 5 parts to 10 parts of the KCl, 10 parts to 20 parts of the molasses, 40 parts to 60 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃.

The present disclosure provides a method for in-situ removal of reservoir plugging using microorganisms and reservoir oil displacement, including the following steps: injecting a microbial mixture into a target reservoir to allow in-situ activation of microorganisms, such that magnetic iron nanoparticles are generated in-situ in the target reservoir to obtain a plugging-removed target reservoir; where the microbial mixture includes an iron-reducing bacterium fermentation broth and an iron-reducing bacterium activator; and the iron-reducing bacterium activator includes NH₄Cl, NaCl, KCl, molasses, HCOONa, CoSO₄, Na₂MoO₄, NiCl₂, Na₂WO₄, and H₃BO₃; and injecting water into the plugging-removed target reservoir to allow the reservoir oil displacement. In the present disclosure, the method utilizes the iron-reducing activity of the iron-reducing bacterium in the iron-reducing bacterium fermentation broth. After being activated by the activator, the iron-reducing bacterium can reduce and convert amorphous Fe(OH)₃ in the target reservoir into magnetic iron nanoparticles through its own reductase and active substances. This conversion process is conducted under in-situ on the target reservoir, and can effectively remove the reservoir plugging caused by Fe(OH)₃. Moreover, the magnetic iron nanoparticles produced in situ can effectively conduct oil displacement. Furthermore, the iron-reducing bacterium is used in the present disclosure. Due to a high migration capacity of microorganisms in the formation, the iron-reducing bacterium can enter reservoir areas that chemical reagents cannot reach, and conduct in-situ reduction activities in these areas to produce magnetic iron nanoparticles, thereby effectively increasing the area involved in plugging removal and oil displacement. In summary, the method removes reservoir plugging caused by secondary precipitation of Fe(OH)₃ through an environmental-friendly biological method. Meanwhile, the microorganisms with an iron-reducing activity enable a green and low-cost biological method to produce highly-active magnetic nanoparticles in situ to allow the reservoir oil displacement. In the present disclosure, a microbial activity can simultaneously achieve the dual effects of plugging removal and oil displacement, thereby reducing construction costs and improving construction effects.

At the same time, the method provided by the present disclosure can reduce the toxicity of heavy metals during the production of magnetic iron nanoparticles by microorganisms, such that the generated nanoparticles can exhibit potential natural nano-activity. The microbial inherent reductase and active substances allow the microorganisms to synthesize a nano-oil displacement agent (magnetic iron nanoparticles) without the participation of organic solvents, stabilizers and other chemical substances. As a result, the microbial synthesis of nano-oil displacement agent in the method is simple, rapid, non-toxic, and ecological-friendly.

The present disclosure further provides an iron-reducing bacterium activator, including the following components in parts by mass: 10 parts to 15 parts of the NH₄Cl, 5 parts to 10 parts of the NaCl, 5 parts to 10 parts of the KCl, 10 parts to 20 parts of the molasses, 40 parts to 60 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃. In the present disclosure, the iron-reducing bacterium activator can effectively activate the iron-reducing activity of the iron-reducing bacterium in a high-pressure environment of the target reservoir (at 30° C. to 45° C. under 5 MPa to 20 MPa), thereby achieving the dual effects of in-situ plugging removal and oil displacement. Moreover, the components of the iron-reducing bacterium activator are all biodegradable components, thus achieving environmental friendliness during the plugging removal and oil displacement.

### BRIEF DESCRIPTION OF THE DRAWINGS

FIG. 1 shows an electron microscope photograph of the iron-reducing bacterium used in Example 1 of the present disclosure;

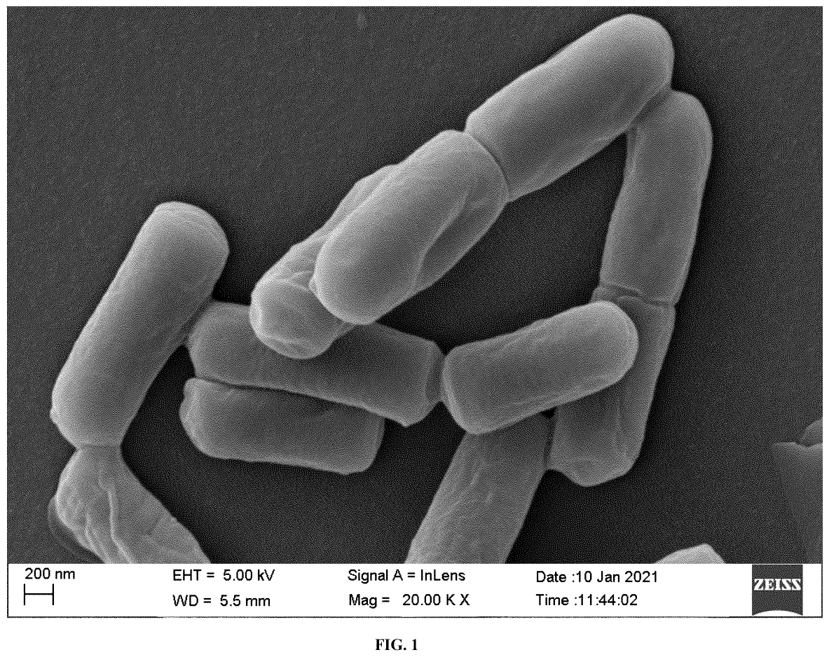

FIG. 2 shows an electron microscope photograph of Fe(OH)₃ blockage in the target reservoir in Example 1 of the present disclosure;

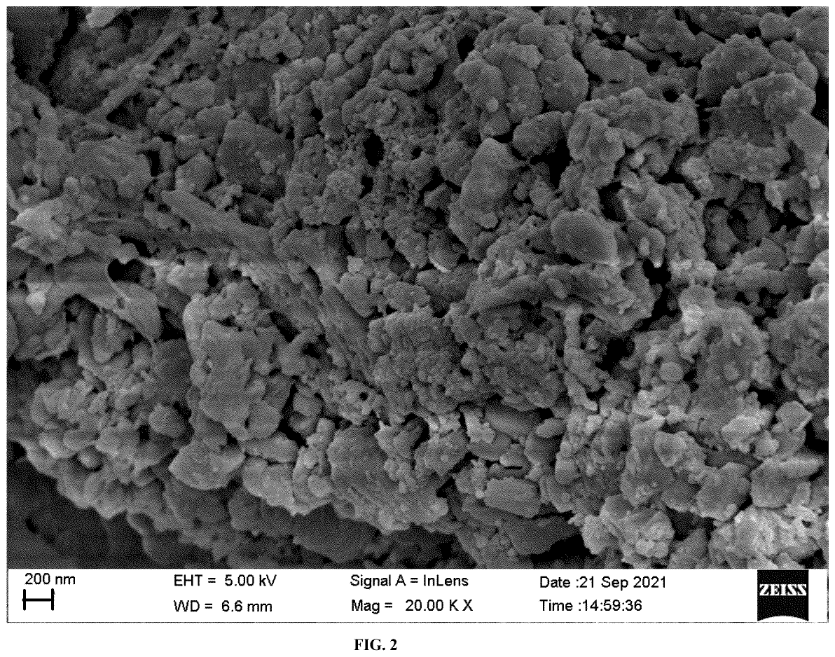

FIG. 3 shows an electron microscope photograph of magnetic iron nanoparticles produced in situ by the iron-reducing bacterium in Example 1 of the present disclosure; and

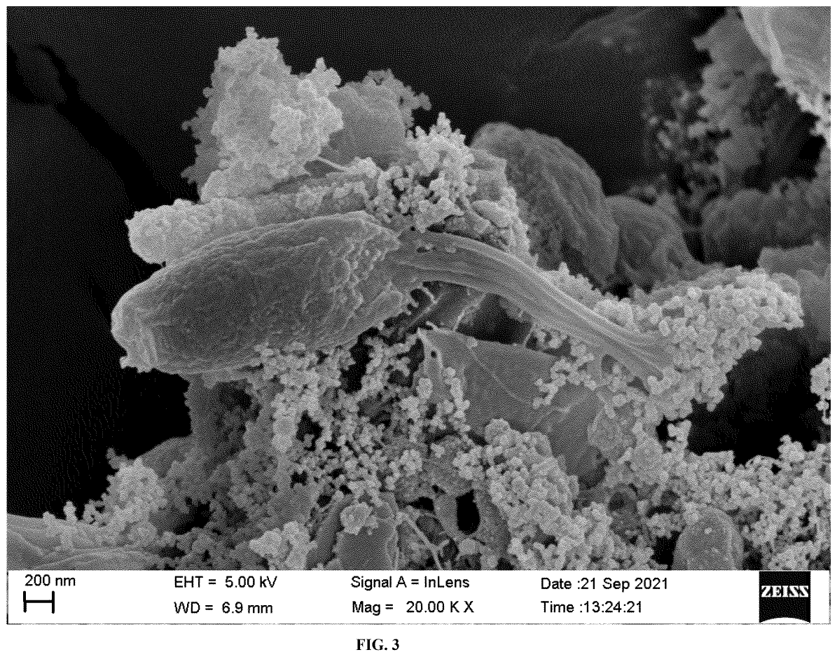

FIG. 4 shows an electron microscope photograph of the Fe(OH)₃ blockage transformed into the magnetic iron nanoparticles after activation in Example 1 of the present disclosure.

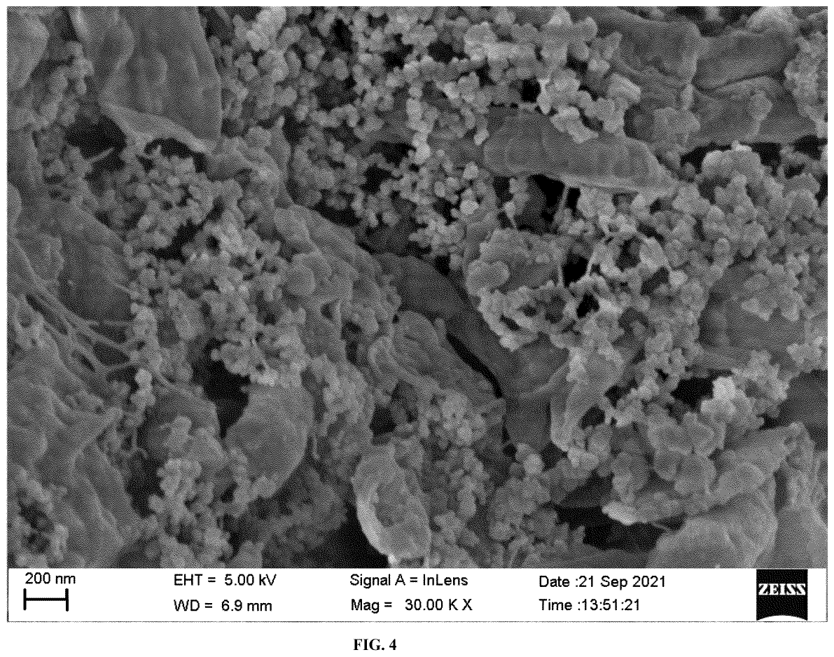

### DETAILED DESCRIPTION OF THE EMBODIMENTS

The present disclosure provides a method for in-situ removal of reservoir plugging using microorganisms and in-situ reservoir oil displacement, including the following steps:
injecting a microbial mixture into a target reservoir to allow in-situ activation of microorganisms, such that magnetic iron nanoparticles are generated in-situ in the target reservoir to obtain a plugging-removed target reservoir; where the microbial mixture includes an iron-reducing bacterium fermentation broth and an iron-reducing bacterium activator; and the iron-reducing bacterium activator includes NH₄Cl, NaCl, KCl, molasses, HCOONa, CoSO₄, Na₂MoO₄, NiCl₂, Na₂WO₄, and H₃BO₃; and
injecting water into the plugging-removed target reservoir to allow the reservoir oil displacement.

In the present disclosure, unless otherwise specified, all raw materials for preparation are commercially available products well known to those skilled in the art.

In the present disclosure, a microbial mixture is injected into a target reservoir to allow in-situ activation of microorganisms, such that magnetic iron nanoparticles are generated in-situ in the target reservoir to obtain a plugging-removed target reservoir; where the microbial mixture includes an iron-reducing bacterium fermentation broth and an iron-reducing bacterium activator; and the iron-reducing bacterium activator includes NH₄Cl, NaCl, KCl, molasses, HCOONa, CoSO₄, Na₂MoO₄, NiCl₂, Na₂WO₄, and H₃BO₃.

In the present disclosure, the target reservoir is an iron hydroxide-plugged reservoir or an iron-containing mineral-rich oil reservoir.

In a specific example of the present disclosure, the target reservoir has an average porosity of 14.9% and an average permeability of 26.88×10⁻³ μm². Surface crude oil has a density of 0.8629 kg/m³ and a viscosity of 20.15 mPa·S. The target reservoir has an oil field water total salinity of 14.18×10⁴ mg/L and an average pH value of 8.3; and oil field water in the target reservoir belongs to CaCl₂ type.

In the present disclosure, the iron-reducing bacterium fermentation broth includes an iron-reducing bacterium; and the iron-reducing bacterium is one or more selected from the group consisting of Shewanella chilikensis, Bacillus alkalitelluris, Tessaracoccus profundi, Tessaracoccus oleiagri, Clostridium swellfunianum, and Deferribacter thermophilus; more preferably one or more selected from the group consisting of Shewanella Tessaracoccus oleiagri, Clostridium swellfunianum, Bacillus alkalitelluris, and Deferribacter thermophilus.

As one or more examples of the present disclosure, the iron-reducing bacterium is preferably the Shewanella chilikensis.

As one or more examples of the present disclosure, the iron-reducing bacterium preferably includes the Tessaracoccus oleiagri and the Clostridium swellfunianum.

As one or more examples of the present disclosure, the iron-reducing bacterium preferably includes the Shewanella chilikensis, the Bacillus alkalitelluris, and the Deferribacter thermophilus.

In the present disclosure, the iron-reducing bacterium in the iron-reducing bacterium fermentation broth has a bacterial count of preferably 5×10⁷ cells/mL to 8×10⁷ cells/mL, more preferably 5.7×10⁷ cells/mL to 7.5×10⁷ cells/mL, specifically preferably 5.7×10⁷ cells/mL, 7.5×10⁷ cells/mL, or 6.8×10⁷ cells/mL.

In the present disclosure, a preparation process of the iron-reducing bacterium fermentation broth includes the following steps:
inoculating the iron-reducing bacterium into a medium with an inoculum size of preferably 5% to allow anaerobic fermentation at 37° C. for 5 d to obtain the iron-reducing bacterium fermentation broth; where the medium includes corn steep liquor, molasses, HCOONa, (NH₄)₂HPO₄, and NaCl.

In the present disclosure, in the medium, a dry weight of the corn steep liquor has a mass percent of preferably 3%; the molasses has a mass percent of preferably 3%; the HCOONa has a mass percent of preferably 5%; the (NH₄)₂HPO₄ has a mass percent of preferably 0.1%; the NaCl has a mass percent of preferably 1%; and the medium has a pH value of preferably 7.2.

In the present disclosure, the iron-reducing bacterium activator preferably includes the following components in parts by mass: 10 parts to 15 parts of the NH₄Cl, 5 parts to 10 parts of the NaCl, 5 parts to 10 parts of the KCl, 10 parts to 20 parts of the molasses, 40 parts to 60 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃.

As one or more examples of the present disclosure, the iron-reducing bacterium activator preferably includes the following components in parts by mass: 10 parts of the NH₄Cl, 5 parts of the NaCl, 5 parts of the KCl, 15 parts of the molasses, 55 parts of the HCOONa, 2 parts of the CoSO₄·7H₂O, 2 parts of the Na₂MoO₄·2H₂O, 2 parts of the NiCl₂·6H₂O, 2 parts of the Na₂WO₄·2H₂O, and 2 parts of the H₃BO₃.

As one or more examples of the present disclosure, the iron-reducing bacterium activator preferably includes the following components in parts by mass: 15 parts of the NH₄Cl, 5 parts of the NaCl, 5 parts of the KCl, 20 parts of the molasses, 50 parts of the HCOONa, 1 part of the CoSO₄·7H₂O, 1 part of the Na₂MoO₄·2H₂O, 1 part of the NiCl₂·6H₂O, 1 part of the Na₂WO₄·2H₂O, and 1 part of the H₃BO₃.

As one or more examples of the present disclosure, the iron-reducing bacterium activator preferably includes the following components in parts by mass: 10 parts of the NH₄Cl, 7 parts of the NaCl, 7 parts of the KCl, 16 parts of the molasses, 50 parts of the HCOONa, 2 parts of the CoSO₄·7H₂O, 2 parts of the Na₂MoO₄·2H₂O, 2 parts of the NiCl₂·6H₂O, 2 parts of the Na₂WO₄·2H₂O, and 2 parts of the H₃BO₃.

In the present disclosure, the microbial mixture preferably further includes water; and the water serves as a diluent of the microbial mixture.

In the present disclosure, the microbial mixture includes preferably 5% to 10% of the iron-reducing bacterium fermentation broth and preferably 5% to 10% of the iron-reducing bacterium activator by mass percent.

In the present disclosure, the microbial mixture is preferably injected through a water injection well in the mining area.

In the present disclosure, the microbial mixture is preferably subjected to one injection or multiple equal injections, and more preferably one injection or two equal injections.

As one or more examples of the present disclosure, the microbial mixture is subjected to the two equal injections, and the two equal injections have an interval of preferably 7 d. Each injection is conducted for 2 d.

In the present disclosure, the microbial mixture is injected at an injection volume of preferably 15 PV to 20 PV; the PV represents a pore volume, and a calculation formula of the PV is shown in Formula 1:
1PV=π×R²×H×Ø Formula 1; in Formula 1, R represents a treatment radius of the microbial mixture, in m; H represents a thickness of a water-absorbing layer, in m; and Ø represents an average porosity of a formation.

In the present disclosure, the water-absorbing layer is a water-absorbing layer in the target reservoir. The formation average porosity refers to a formation average porosity of the target reservoir.

In the present disclosure, the microbial mixture is injected at an injection rate of preferably (0.1-0.5) m³/min, more preferably (0.15-0.45) m³/min.

In the present disclosure, the microbial mixture is injected at a pressure of less than or equal to 20 MPa.

In the present disclosure, after the microbial mixture is injected into the target reservoir, the water injection well used for injection is preferably closed to allow the in-situ activation of microorganisms.

In the present disclosure, the in-situ activation of the microorganism is conducted for preferably 7 d to 10 d, more preferably 7 d to 8 d.

In the present disclosure, during the in-situ activation of the microorganisms, the iron-reducing bacterium in the iron-reducing bacterium fermentation broth exert iron-reducing activity under an action of the iron-reducing bacterium activator, such that the iron-reducing bacterium can reduce and convert amorphous Fe(OH)₃ in the target reservoir into magnetic iron nanoparticles through its own reductase and active substances.

In the present disclosure, water is injected into the plugging-removed target reservoir to allow the reservoir oil displacement.

In the present disclosure, the water is preferably injected through a water injection well.

In the present disclosure, the water is injected at a flow rate of preferably (0.1-0.5) m³/min, more preferably (0.15-0.45) m³/min.

The present disclosure further provides an iron-reducing bacterium activator, including the following components in parts by mass: 10 parts to 15 parts of the NH₄Cl, 5 parts to 10 parts of the NaCl, 5 parts to 10 parts of the KCl, 10 parts to 20 parts of the molasses, 40 parts to 60 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃, preferably 10 parts to 15 parts of the NH₄Cl, 5 parts to 7 parts of the NaCl, 5 parts to 7 parts of the KCl, 15 parts to 20 parts of the molasses, 50 parts to 55 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃.

In order to further illustrate the present disclosure, the technical solutions provided by the present disclosure are described in detail below in connection with accompanying drawings and examples, but these examples should not be understood as limiting the claimed scope of the present disclosure.

### Example 1

In this example, an iron-reducing bacterium in the iron-reducing bacterium fermentation broth was Shewanella chilikensis.

The Shewanella chilikensis was inoculated into a medium to allow anaerobic fermentation (at an inoculum size of 5%) to obtain a Shewanella chilikensis fermentation broth; where the medium included: a corn steep liquor dry powder 3%, molasses 3%, HCOONa 5%, (NH₄)₂HPO₄ 0.1%, and NaCl 1%, with a pH value of 7.2; the inoculum size of the Shewanella chilikensis was 5%, the anaerobic fermentation was conducted at 37° C. for 5 d; and the Shewanella chilikensis fermentation broth had a bacterial concentration of 6.8×10⁷ cells/mL.

In this example, the iron-reducing bacterium activator included the following components in parts by mass: 10 parts of NH₄Cl, 5 parts of NaCl, 5 parts of KCl, 15 parts of molasses, 55 parts of HCOONa, 2 parts of CoSO₄·7H₂O, 2 parts of Na₂MoO₄·2H₂O, 2 parts of NiCl₂·6H₂O, 2 parts of Na₂WO₄·2H₂O, and 2 parts of H₃BO₃.

In this example, a core displacement experiment is conducted to simulate an in-situ magnetic nano-oil displacement effect of actual reservoirs, including:

1) Preparation of an artificial core: in order to simulate Fe(OH)₃ core blockage, Fe(OH)₃ accounting for 30% of a clay mineral mass fraction was added to the clay mineral by adsorption method to obtain the artificial core containing Fe(OH)₃ blockage.

2) Saturated crude oil treatment of cores: the artificial core was dried at 120° C. for 24 h. A thermostat of the displacement device was turned on and set to 55° C., the dried artificial core was put into a core holder and vacuumized for 4 h while maintaining an annular pressure at 3 MPa to 4 MPa. Simulated formation water was injected at a constant flow rate of 0.5 mL/min until a displacement pressure difference reached equilibrium at both ends of the core. 7 PV crude oil was injected at a constant flow rate of 0.2 mL/min, 5 PV crude oil was injected at a constant flow rate of 0.3 mL/min, 3 PV crude oil was injected at a constant flow rate of 0.5 mL/min, and a gate valve was closed to allow standing at constant temperature for 24 h.

3) An iron-reducing bacterium activator and the Shewanella chilikensis fermentation broth were added into 1 L of water to obtain a microbial mixture, where the microbial mixture included 10% of the iron-reducing bacterium activator and 10% of the Shewanella chilikensis fermentation broth by mass percent. The microbial mixture was transferred to an intermediate container for the core experiment, and N₂ was introduced to remove oxygen in the intermediate container.

4) First formation water flooding: an advection pump was turned on, a valve corresponding to the simulated formation water intermediate container was opened, and simulating formation water flooding was started with an injection flow rate of 0.3 mL/min. A produced oil-water mixture was collected, and the formation water flooding was stopped when a water content in the displacement-produced fluid reached 98%. An injection pressure difference at both ends of the core was recorded, and the amount of crude oil displacement was measured.

5) The advection pump was turned on, the corresponding valve of the intermediate container of the microbial mixture formed by the iron-reducing bacterium fermentation broth and the iron-reducing bacterium activator was opened, and 2 PV of the corresponding fluid was injected at an injection flow rate of 0.1 mL/min. All gate valves were closed, a growth environment of microbial formation conditions was simulated with an environment of the core in the holder, and the well was shut down at a constant temperature of 55° C. for 10 d.

6) Second formation water flooding: the advection pump was turned on, the valve corresponding to the simulated formation water intermediate container was opened, and second simulating formation water flooding was started with an injection flow rate of 0.3 mL/min. A produced oil-water mixture was collected, and the formation water flooding was stopped when a water content in the displacement-produced fluid reached 98%. An injection pressure difference at both ends of the core was recorded, and the amount of crude oil displacement was measured.

### Example 2

A method of this example was basically the same as that of Example 1, except that: (1) the iron-reducing bacterium in the iron-reducing bacterium fermentation broth in this example included Tessaracoccus oleiagri and Clostridium swellfunianum (the two bacteria in the iron-reducing bacterium fermentation broth were mixed in an equal proportion). After fermentation, the iron-reducing bacterium fermentation broth had a total bacterial concentration of 7.5×10⁷ cells/mL.

(2) The iron-reducing bacterium activator included the following components in parts by mass: 15 parts of NH₄Cl, 5 parts of NaCl, 5 parts of KCl, 20 parts of molasses, 50 parts of HCOONa, 1 part of CoSO₄·7H₂O, 1 part of Na₂MoO₄·2H₂O, 1 part of NiCl₂·6H₂O, 1 part of Na₂WO₄2H₂O, and 1 part of H₃BO₃.

### Example 3

A method of this example was basically the same as that of Example 1, except that: (1) the iron-reducing bacterium in the iron-reducing bacterium fermentation broth in this example included Shewanella chilikensis, Bacillus alkalitelluris, and Deferribacter thermophilus (the two bacteria in the iron-reducing bacterium fermentation broth were mixed in an equal proportion). After fermentation, the iron-reducing bacterium fermentation broth had a total bacterial concentration of 5.7×10⁷ cells/mL.

(2) The iron-reducing bacterium activator included the following components in parts by mass: 10 parts of NH₄Cl, 7 parts of NaCl, 7 parts of KCl, 16 parts of molasses, 50 parts of HCOONa, 2 parts of CoSO₄·7H₂O, 2 parts of Na₂MoO₄·2H₂O, 2 parts of NiCl₂·6H₂O, 2 parts of Na₂WO₄·2H₂O, and 2 parts of H₃BO₃.

In the present disclosure, a core displacement evaluation experiment was conducted on the microbial in-situ production of nano-plugging removal and oil displacement in Examples 1 to 3. A pressure difference between the two ends of the core, a core permeability, and a recovery factor before and after in-situ nano-plugging removal and oil displacement were shown in Table 1.

TABLE 1
Core displacement evaluation results of microbial in-situ production
of nano-plugging removal and oil displacement in Examples 1 to 3:

| | First formation water flooding | | | Second formation water flooding | | |
|---|---|---|---|---|---|---|
| | Injection | | | Injection | | Improved |
| | pressure | | Recovery | pressure | | recovery |
| Item | difference | Permeability | factor | difference | Permeability | factor |
| Core | (MPa) | mD | % | (MPa) | mD | % |
| Example 1 | 0.035 | 15.72 | 43.29 | 0.021 | 26.29 | 16.53 |
| Example 2 | 0.042 | 13.10 | 45.31 | 0.024 | 22.93 | 15.29 |
| Example 3 | 0.045 | 12.23 | 40.61 | 0.017 | 36.68 | 20.65 |

As shown in Table 1: the present disclosure provided a new technology of in-situ nano-plugging removal and oil displacement. After microbial in-situ nano-plugging removal in the core simulation experiment, the injection pressure difference at both ends of the core decreased to varying degrees, and the core permeability increased to varying degrees. Specifically, the maximum pressure difference across the core was reduced by 62.2%, while the core permeability was increased from the original 12.23 mD to 36.68 mD (Example 3). These examples showed that microbial in-situ nano-production treatment could effectively relieve core plugging, reduce injection pressure, and increase core permeability. In the core simulation experiment, after microbial in-situ nano-production treatment, the core crude oil recovery factor also increased to varying degrees, with a recovery factor increased by 15.29 to 20.65%. This indicated that microbial in-situ nano-production treatment could effectively improve crude oil recovery factor.

### Use Example 1

QGQ6-29 was a well group in the QHQGQ oil field. Production layers: oil field reservoirs were distributed in N₂², N¹, E₃², and E₃¹, with burial depths of 460 m to 1,926 m, and the oil well section was about 750 m long; where E₃² and E₃¹ were the main oil storage layers of the oil field. The reservoir had poor physical properties, with an average porosity of 14.9% and an average permeability of 26.88×10⁻³ μm². Surface crude oil had a density 0.8629 kg/m³ and a viscosity of 20.15 mPa·S. The reservoir had an oil field water total salinity of 14.18×10⁴ mg/L and an average pH value of 8.3; and oil field water belonged to CaCl₂ type.

The production of this well group showed the following problems: poor physical properties of the reservoir, prominent conflicts between layers, and difficulty in seepage of injected water in porous media; a comprehensive water content of the oil well changed from 30.42% to 97.33%, and the plane water injection and oil displacement were uneven; it indicated that water flooding on the plane was uneven, with more residual oil at the bottom and poor physical properties in the structure; the injection horizontal sweep efficiency was poor, the sweep coefficient was low, the plane contradictions of the well group were prominent, the water content around the well group was different, and the plane residual oil reserves were relatively large, showing a better potential for remaining oil exploration.

According to the oil and water characteristics of the oil field and the development issues of the QGQ6-29 well group, the method provided in Example 1 was selected to conduct a mine experiment of the QGQ6-29 well group by the microbial in-situ nano-plugging removal and oil displacement.

10 tons of the iron-reducing bacterium fermentation broth in Example 1 was prepared. The iron-reducing bacterium in the iron-reducing bacterium fermentation broth was Shewanella chilikensis. The fermentation of the iron-reducing bacterium fermentation broth included: anaerobic fermentation; components: corn steep liquor dry powder 3%, molasses 3%, HCOONa 5%, (NH₄)₂HPO₄ 0.1%, and NaCl 1%, pH=7.2; an inoculum size of 5%, and fermentation at 37° C. for 5 d.

30 tons of the iron-reducing bacterium activator in Example 1 was prepared. The iron-reducing bacterium activator included: 10 parts of NH₄Cl, 5 parts of NaCl, 5 parts of KCl, 15 parts of molasses, 55 parts of HCOONa, 2 parts of CoSO₄·7H₂O, 2 parts of Na₂MoO₄·2H₂O, 2 parts of NiCl₂·6H₂O, 2 parts of Na₂WO₄2H₂O, and 2 parts of H₃BO₃.

The experiment was conducted in 2 rounds, once every 7 d, with each injection taking 2 d, and a construction period was 20 d in total. The iron-reducing bacterium fermentation broth and the iron-reducing bacterium activator were injected from a wellhead at an injection pressure of not greater than 20 MPa. The specific well group amount, number of slugs, and rounds were shown in Table 2.

TABLE 2
Specific well group amount, number of slugs, and rounds:

| | First round of construction | | | | Second round of construction | | | |
|---|---|---|---|---|---|---|---|---|
| | | Iron- | | | | Iron- | | |
| | | reducing | Replacement | | Iron- | reducing | | Replacement |
| | Iron-reducing | bacterium | with | Well | reducing | bacterium | Well | with |
| Item | bacterium | fermentation | clean | shut- | bacterium | fermentation | shut- | clean |
| Reagent | activator/water | broth | water | down | activator/water | broth | down | water |
| Volume | 5 t/ 45 t | 5 t | 10 t | Day | 8 t/80 t | 8 t | 10 t | Day 7 |
| | | | | 7 | | | | |

The construction period was 20 d in total. After the construction was completed, QGQ6-29 resumed normal water injection production and began to take effect after 35 d, with a cumulative oil increase of 968 m³ during the validity period.

In summary, compared with the prior art, the method provided by the present disclosure has the following advantages:

(1) The method uses the iron-reducing activity of microorganisms to remove the plugging caused by Fe (OH) 3 secondary precipitation in the reservoir through an environmental-friendly biological process.

(2) The microorganisms with an iron-reducing nano-production activity enable a green and low-cost biological method to produce highly-active magnetic nanoparticles in situ to allow the reservoir oil displacement.

(3) Due to a high migration capacity of microorganisms in the formation, the microorganisms can enter reservoir areas that chemical reagents cannot reach, and conduct in-situ reduction biological activities in these areas, thereby effectively increasing the area involved in plugging removal and oil displacement.

(4) A microbial activity can simultaneously achieve the dual effects of plugging removal and oil displacement, thereby reducing construction costs and improving construction effects.

(5) The reagents used are all biodegradable products, thus achieving environmental friendliness during the plugging removal and oil displacement.

Although the present disclosure is described in detail in conjunction with the foregoing examples, they are only a part of, not all of, the examples of the present disclosure. Other examples can be obtained based on these examples without creative efforts, and all of these examples shall fall within the protection scope of the present disclosure.

1. A method for in-situ removal of reservoir plugging using microorganisms and in-situ reservoir oil displacement, comprising the following steps:
injecting a microbial mixture into a target reservoir to allow in-situ activation of microorganisms, such that magnetic iron nanoparticles are generated in-situ in the target reservoir to obtain a plugging-removed target reservoir; wherein the microbial mixture comprises an iron-reducing bacterium fermentation broth and an iron-reducing bacterium activator; and the iron-reducing bacterium activator comprises NH₄Cl, NaCl, KCl, molasses, HCOONa, CoSO₄, Na₂MoO₄, NiCl₂, Na₂WO₄, and H₃BO₃; and
injecting water into the plugging-removed target reservoir to allow the reservoir oil displacement.

2. The method according to claim 1, wherein the iron-reducing bacterium fermentation broth comprises an iron-reducing bacterium; and
the iron-reducing bacterium is one or more selected from the group consisting of Shewanella chilikensis, Bacillus alkalitelluris, Tessaracoccus profundi, Tessaracoccus oleiagri, Clostridium swellfunianum, and Deferribacter thermophilus.

3. The method according to claim 1, wherein the iron-reducing bacterium activator comprises the following components in parts by mass:
10 parts to 15 parts of the NH₄Cl, 5 parts to 10 parts of the NaCl, 5 parts to 10 parts of the KCl, 10 parts to 20 parts of the molasses, 40 parts to 60 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃.

4. The method according to claim 1, wherein a preparation process of the iron-reducing bacterium fermentation broth comprises the following steps:
inoculating the iron-reducing bacterium into a medium with an inoculum size of 5% to allow anaerobic fermentation at 37° C. for 5 d to obtain the iron-reducing bacterium fermentation broth; wherein the medium comprises corn steep liquor, molasses, HCOONa, (NH₄)₂HPO₄, and NaCl.

5. The method according to claim 1, wherein the iron-reducing bacterium in the iron-reducing bacterium fermentation broth has a bacterial count of 5×10⁷ cells/mL to 8×10⁷ cells/mL; and
the microbial mixture comprises 5% to 10% of the iron-reducing bacterium fermentation broth and 5% to 10% of the iron-reducing bacterium activator by mass percent.

6. The method according to claim 4, wherein in the medium, a dry weight of the corn steep liquor has a mass percent of 3%; the molasses has a mass percent of 3%; the HCOONa has a mass percent of 5%; the (NH₄)₂HPO₄ has a mass percent of 0.1%; the NaCl has a mass percent of 1%; and the medium has a pH value of 7.2.

7. The method according to claim 1, wherein the microbial mixture is injected at an injection volume of 15 PV to 20 PV; the PV represents a pore volume, and a calculation formula of the PV is shown in Formula 1:
1PV=π×R²×H×Ø Formula 1; in Formula 1, R represents a treatment radius of the microbial mixture, in m; H represents a thickness of a water-absorbing layer, in m; and Ø represents an average porosity of a formation.

8. The method according to claim 1, wherein the microbial mixture is injected at an injection rate of (0.1-0.5) m³/min; and
the in-situ activation of the microorganism is conducted for 7 d to 10 d.

9. The method according to claim 1, wherein the microbial mixture is injected at a pressure of less than or equal to 20 MPa.

10. The method according to claim 2, wherein the iron-reducing bacterium is the Shewanella chilikensis, or comprises the Tessaracoccus oleiagri and the Clostridium swellfunianum, or comprises the Shewanella chilikensis, the Bacillus alkalitelluris, and the Deferribacter thermophilus.

11. The method according to claim 1, wherein the water is injected at a flow rate of (0.1-0.5) m³/min.

12. The method according to claim 11, wherein the water is injected through a water injection well.

13. An iron-reducing bacterium activator, comprising the following components in parts by mass:
10 parts to 15 parts of the NH₄Cl, 5 parts to 10 parts of the NaCl, 5 parts to 10 parts of the KCl, 10 parts to 20 parts of the molasses, 40 parts to 60 parts of the HCOONa, 1 part to 2 parts of CoSO₄·7H₂O, 1 part to 2 parts of Na₂MoO₄·2H₂O, 1 part to 2 parts of NiCl₂·6H₂O, 1 part to 2 parts of Na₂WO₄·2H₂O, and 1 part to 2 parts of the H₃BO₃.

14. The method according to claim 2, wherein a preparation process of the iron-reducing bacterium fermentation broth comprises the following steps:
inoculating the iron-reducing bacterium into a medium with an inoculum size of 5% to allow anaerobic fermentation at 37° C. for 5 d to obtain the iron-reducing bacterium fermentation broth; wherein the medium comprises corn steep liquor, molasses, HCOONa, (NH₄)₂HPO₄, and NaCl.

15. The method according to claim 2, wherein the iron-reducing bacterium in the iron-reducing bacterium fermentation broth has a bacterial count of 5×10₇ cells/mL to 8×10₇ cells/mL; and
the microbial mixture comprises 5% to 10% of the iron-reducing bacterium fermentation broth and 5% to 10% of the iron-reducing bacterium activator by mass percent.

16. The method according to claim 3, wherein the iron-reducing bacterium in the iron-reducing bacterium fermentation broth has a bacterial count of 5×10₇ cells/mL to 8×10₇ cells/mL; and
the microbial mixture comprises 5% to 10% of the iron-reducing bacterium fermentation broth and 5% to 10% of the iron-reducing bacterium activator by mass percent.

17. The method according to claim 14, wherein in the medium, a dry weight of the corn steep liquor has a mass percent of 3%; the molasses has a mass percent of 3%; the HCOONa has a mass percent of 5%; the (NH₄)₂HPO₄ has a mass percent of 0.1%; the NaCl has a mass percent of 1%; and the medium has a pH value of 7.2.

18. The method according to claim 7, wherein the microbial mixture is injected at an injection rate of (0.1-0.5) m₃/min; and
the in-situ activation of the microorganism is conducted for 7 d to 10 d.
